(19) United States
(12) Patent Application Publication
(10) Pub. No.: US 2009/0263332 A1
(43) Pub. Date: Oct. 22, 2009

(54) POLYMER PARTICLE CONTAINING PARAMAGNETIC METAL COMPOUND

(75) Inventors: Takashi TAMURA, Kanagawa (JP); Kazuya TAKEUCHI, Kanagawa (JP); Kazuhiro AIKAWA, Kanagawa (JP)

Correspondence Address: YOUNG & THOMPSON, 209 Madison Street, Suite 500, ALEXANDRIA, VA, 22314, US

(73) Assignee: FUJIFILM CORPORATION, Tokyo (JP)

(21) Appl. No.: 12/426,432

(22) Filed: Apr. 20, 2009

(30) Foreign Application Priority Data
Apr. 21, 2008 (JP) 2008-110049

(51) Int. Cl. A61B 5/055

(52) U.S. Cl. 424/9.361; 424/9.3

(57) ABSTRACT

A magnetic resonance imaging agent which includes: a polymer containing the structural unit represented by the following formula (I) and the structural unit represented by the following formula (II) in a molar ratio of 5 to 80:20 to 95; a paramagnetic metal compound; and a ligand molecule:

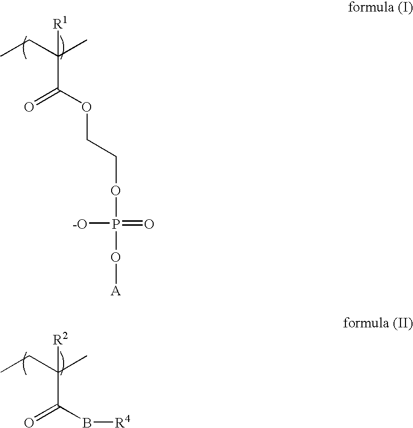

wherein R¹ represents hydrogen atom or methyl group; R² represents a hydrogen atom or a methyl group; A represents —(CH₂)₂N⁺(CH₃)₃ or the like; and B represents oxygen atom, sulfur atom, —CH₂—, or —NH—; and R⁴ represents hydrogen atom, an optionally substituted alkyl group, or an optionally substituted aryl group, which affords good retention in the blood and a good ability to accumulate in diseased areas.

### CROSS-REFERENCE TO RELATED APPLICATIONS

This application claims benefit of priority under 35 USC 119 to Japanese Patent Application No. 2008-110049 filed on Apr. 21, 2008, the disclosure of which is expressly incorporated by reference herein in its entirety.

### TECHNICAL FIELD

The present invention relates to a magnetic resonance imaging agent containing a phospholipid-like polymer and a paramagnetic metal compound

### BACKGROUND ART

A major example of non-invasive method for diagnosing arteriosclerosis includes X-ray angiography. This method contrasts vascular flows by using a water-soluble iodine-containing contrast medium, and therefore, the method has a problem of difficulty in distinguishing pathological lesions from normal tissues. By applying the above method, only a pathological lesion where constriction progresses 50% or more can be detected, and it is difficult to detect a lesion before onset of attack of an ischemic disease.

As diagnostic methods other than the above, methods of detecting a disease by nuclear magnetic resonance tomography (MRI) using a contrast medium, which is kinetically much distributed in arteriosclerotic plaques, have been reported in recent years. However, all the compounds reported as the contrast medium have a problem for use in the diagnostic methods. For example, hematoporphyrin derivatives (see, U.S. Pat. No. 4,577,636, the disclosure of which is expressly incorporated by reference herein in its entirety) are pointed out to have a defect of, for example, dermal deposition and coloring of skin. As for gadolinium complexes having a perfluorinated side chain, which have been reported to accumulate in lipid-rich plaques (see, Circulation, 109, 2890, 2004, the disclosure of which is expressly incorporated by reference herein in its entirety), accumulation in lipid-rich tissues and organs in vivo, such as fatty livers, renal epitheliums, and tendons of muscular tissues is of concern.

Currently, one of the gadolinium complexes that are widely employed as magnetic resonance imaging agents is a gadolinium complex of diethylenetriaminepentaacetic acid (DTPA). Although the complex is characterized by low toxicity, the complex has a short retention time in the blood and is rapidly expelled, making it difficult to selectively image sites of disease.

Accordingly, there are reports of attempts to selectively image tissue by enclosing a paramagnetic metal compound in a liposome to enhance retention in the blood. However, the operation of creating a supercritical state and the like to increase the quantity of paramagnetic metal compound enclosed has proven quite complex (see Japanese Unexamined Patent Publication (KOKAI) No. 2006-45132, the disclosure of which is expressly incorporated by reference herein in its entirety).

Phospholipid-mimicking compounds in which a phosphatidylethanolamine (PE) having two fatty acid esters is amide bonded to diethylenetriaminepentaacetic acid (DTPA) are known (for example: Polymeric Materials Science and Engineering, 89, 148 (2003), the disclosure of which is expressly incorporated by reference herein in its entirety). There are also reports of liposomes of gadolinium complexes of this compound (Inorganica Chimica Acta, 331, 151 (2002), the disclosure of which is expressly incorporated by reference herein in its entirety). However, this complex is not readily soluble, and thus affords poor handling properties in the course of conversion to a liposome. There are also concerns about accumulation within the body, toxicity, and the like.

A separately reported gadolinium complex incorporating a hydrophobic group in the form of a single higher fatty acid ester group (see Japanese Unexamined Patent Publication (KOKAI) No. 2007-091640, the disclosure of which is expressly incorporated by reference herein in its entirety) affords good solubility and can be employed to prepare liposome formulations. However, there is a problem in that only a low concentration of this complex can be introduced into the liposome.

Although there are reports of attempts to selectively image tissue by enclosing a paramagnetic metal compound in a polymer to enhance retention in the blood (for example, see International Patent Publication No. WO01/064164, the disclosure of which is expressly incorporated by reference herein in their entirety), there are concerns about accumulation and toxicity due to the low biocompatibility of polymers.

Further, there are reports of attempts to selectively image tissue by linking the chelation (coordination) site of a paramagnetic metal compound to the main chain of a polymer through a covalent bond to enhance the retention in the blood of the paramagnetic metal compound (for example, see International Patent Publication No. WO96/32967, the disclosure of which is expressly incorporated by reference herein in their entirety), but there are concerns that the paramagnetic metal compound will accumulate within the body over an extended period, and that the metal chelation site will be gradually metabolized, resulting in harm to the body by free metal (ions).

Additionally, there are known substances that mimic biomembranes (cellular membranes). These include 2-methacryloyloxyethylphosphoryl choline (MPC), comprising in a single molecule both a phospholipid polar group (phosphorylcholine group), which is a constituent component of biomembranes, and a methacryloyl group having polymeric properties, as well as MPC polymers, which are copolymers of MPC and methacrylic acid esters (Japanese Patent No. 2,870,727, the disclosure of which is expressly incorporated by reference herein in its entirety). Since MPC polymers have unprecedented high biocompatibility due to extremely low interaction with biocomponents such as proteins and blood cells, exhibit extremely good antithrombotic properties, and the like, a variety of applications is conceivable. However, there has been no report thus far of the application of these compounds with paramagnetic metal compounds as magnetic resonance imaging agents.

### SUMMARY OF THE INVENTION

The object of the present invention is to provide a magnetic resonance imaging agent affording good retention in the blood and a good ability to accumulate in diseased areas.

The present inventors conducted extensive research to achieve the above object, resulting in the discovery that the blood retention of paramagnetic metal compounds was enhanced by enclosing a paramagnetic metal compound in a phospholipid-like polymer in the form of a chemical species that was similar to phospholipids present within the body and that was characterized by the high biocompatibility of phospholipids. The present invention was devised on the basis of this information.

The present invention thus provides [1] to [13] below:

[1] A magnetic resonance imaging agent including: a polymer containing the structural unit represented by the following formula (I) and the structural unit represented by the following formula (II) in a molar ratio of 5 to 80:20 to 95; a paramagnetic metal compound; and a ligand molecule:

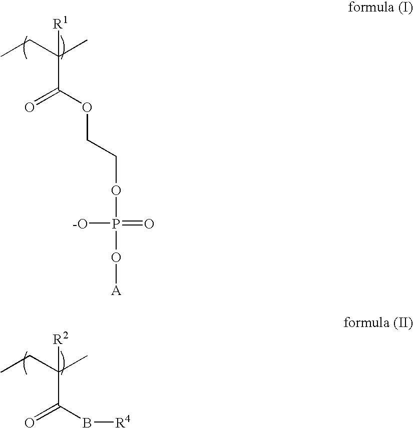

wherein R¹ represents hydrogen atom or methyl group; R² represents a hydrogen atom or a methyl group; A represents a group represented by one of the following formulas:

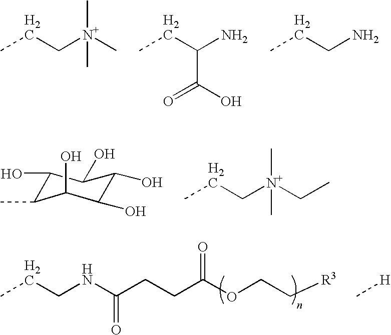

wherein the dotted line represents the O-A bond portion in formula (I); R³ represents hydroxyl group, methyloxy group, ethyloxy group, or phenyloxy group; and n represents an integer of 1 to 100; and B represents oxygen atom, sulfur atom, —CH₂—, or —NH—; and R⁴ represents hydrogen atom, an optionally substituted alkyl group, or an optionally substituted aryl group.

[2] The magnetic resonance imaging agent according to [1], wherein the ligand molecule is the compound represented by general formula (2):

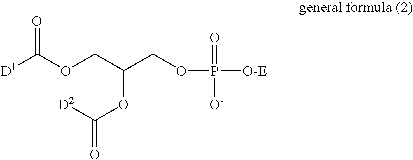

wherein each of D¹ and D² independently represents hydrogen atom, an optionally substituted alkyl group, or an optionally substituted aryl group; and E represents a group represented by one of the following formulas:

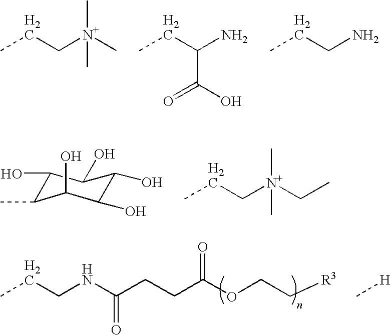

wherein the dotted line represents the O-A bond portion in formula (I); R³ represents hydroxyl group, methyloxy group, ethyloxy group, or phenyloxy group; and n represents an integer of 1 to 100.

[3] The magnetic resonance imaging agent according to [1], wherein the ligand molecule is at least one member selected from the group consisting of: phosphatidic acid, phosphatidylcholine, phosphatidylserine, phosphatidylethanolamine, and phosphatidylinositol.

[4] The magnetic resonance imaging agent according to any one of [1] to [3], wherein the weight ratio of the ligand molecule and the polymer is from 10:90 to 50:50.

[5] The magnetic resonance imaging agent according to any one of [1] to [4], including a particle having a diameter of 4 to 400 nm containing a polymer and a paramagnetic metal compound.

[6] The magnetic resonance imaging agent according to any one of [1] to [5], wherein the polymer is a copolymer of compound A below and an acrylic acid ester or a methacrylic acid ester.

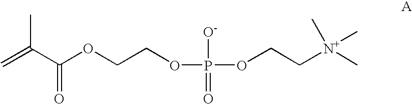

[7] The magnetic resonance imaging agent according to any one of [1] to [6], wherein the paramagnetic metal compound is iron oxide or a metal complex compound.

[8] The magnetic resonance imaging agent according to any one of [1] to [6], wherein the paramagnetic metal compound is a gadolinium metal complex compound.

[9] The magnetic resonance imaging agent according to any one of [1] to [8], used to image localized tissue or a diseased area in which the presence of macrophages or smooth muscle cells is pronounced.

[10] The magnetic resonance imaging agent according to [9], wherein the localized tissue or diseased area in which the presence of macrophages or smooth muscle cells is pronounced is selected from the group consisting of a tumor, a site of inflammation, or a site of infection.

[11] The magnetic resonance imaging agent according to any one of [1] to [8], used to image vascular disease.

[12] The magnetic resonance imaging agent according to any one of [1] to [8], used to image an arteriosclerotic lesion.

From another aspect, the present invention provides the use of the above polymer, paramagnetic metal compound, and ligand molecule to prepare the imaging agent of [1] to [8] above; and provides an imaging method comprising the step of conducting imaging after administering a particle containing the above polymer, a paramagnetic metal compound, and a ligand molecule to a mammal, including a human being.

### MODES OF CARRYING OUT THE INVENTION

The present invention is described in detail below.

In the present Specification, a range expressed as a pair of numbers separated by the word “to” includes the preceding and succeeding numbers as lower and upper limits, respectively.

The magnetic resonance imaging agent of the present invention includes a polymer containing the structural unit represented by formula (I) above and the structural unit represented by formula (II) above as repeating units. This polymer can be obtained by copolymerizing the monomer represented by formula (I′) below and the monomer represented by formula (II′) below.

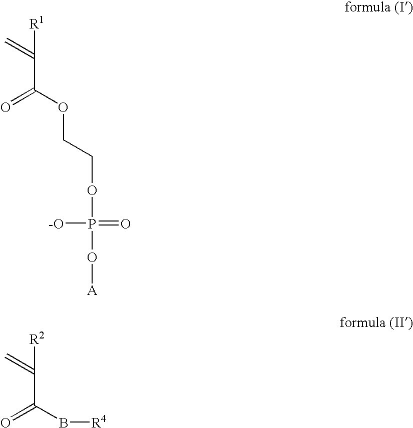

The molar ratio of the structural unit represented by formula (I) and the structural unit represented by formula (II) (number of moles of structural unit (I): number of moles of structural unit II) is 5 to 80:20 to 95, preferably 20 to 40:60 to 80. When the number of moles of the structural unit represented by formula (I) is less than 5 percent of the total number of moles of structural units (I) and (II), the biocompatibility of the polymer may decrease, resulting in a concern of toxicity. When the number of moles of the structural unit represented by formula (I) is 80 percent or greater of the total number of moles of structural units (I) and (II), the polymer becomes excessively hydrophilic, making it impossible to stably maintain the paramagnetic metal compound.

The polymer may be a copolymer obtained by alternating copolymerization, a copolymer obtained by block copolymerization, or a copolymer obtained by random copolymerization of the monomer represented by formula (I′) with the monomer represented by formula (II′).

The molecular weight of the polymer may be 1,000 to 200,000, preferably 1,000 to 100,000, and more preferably, 5,000 to 80,000. When the molecular weight is lower than 1,000, it becomes impossible to stably maintain the paramagnetic metal compound. When the molecular weight exceeds 200,000, there is a possibility of delayed biodegradation of the polymer and delayed discharge of the polymer from the body.

In the structural unit represented by formula (I), R¹ represents hydrogen atom or methyl group, with a methyl group being preferable. In the structural unit represented by formula (II), R² represents hydrogen atom or methyl group, with a methyl group being preferable.

A represents a group represented by one of the following formulas:

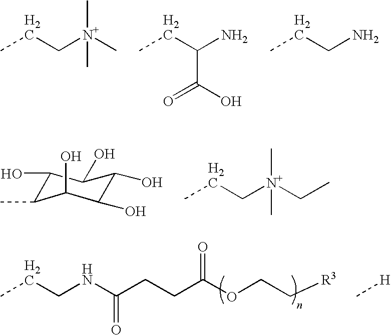

(wherein the dotted line represents the O-A bond portion in formula (I); R³ represents a hydroxyl group, methyloxy group, ethyloxy group, or phenyloxy group; n represents an integer of 1 to 100). A preferably represents the group represented by the following formula.

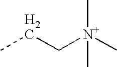

B represents oxygen atom, sulfur atom, —CH₂—, or —NH—.

R⁴ represents hydrogen atom, an optionally substituted alkyl group, or an optionally substituted aryl group. The unsubstituted alkyl group may have a branched structure, may contain an unsaturated group, and may have preferably 1 to 30, more preferably 4 to 20, total carbon atoms. Examples of such alkyl groups include butyl, s-butyl, t-butyl, hexyl, octyl, decyl, tetradecyl, pentadecyl, heptadecyl, cyclohexyl, and cyclohexenyl groups. Among these, preferable examples are decyl, tetradecyl, pentadecyl, heptadecyl, and cyclohexyl groups. Preferable examples include tetradecyl, pentadecyl, and heptadecyl groups. The substituted alkyl group may have a branched structure, may contain an unsaturated group, and may have preferably 1 to 30, more preferably 4 to 25, and most preferably, 10 to 20 total carbon atoms. The substituent in a substituted alkyl group may be a monovalent substituent such as hydroxyl group, an alkoxy group, cyano group, or a halogen atom; or a divalent substituent such as ether bond, sulfide bond, carbonyl group, amide group, urethane group, urea group, or ester group.

The unsubstituted aryl group may have preferably 6 to 30, more preferably 6 to 20, total carbon atoms. Examples of such aryl groups include phenyl, naphthyl, anthracenyl, and pyrenyl groups. The substituent in a substituted aryl group may be a monovalent substituent such as an alkyl group, an aryl group, hydroxyl group, an alkoxy group, cyano group, or a halogen atom; or a divalent substituent such as ether bond, sulfide bond, carbonyl group, amide group, urethane group, urea group, or ester group. The alkyl substituent in a substituted aryl group may be branched, may have a double or triple bond, and may have preferably 1 to 20, more preferably 1 to 6, total carbon atoms. Examples are: methyl, ethyl, ethynyl, propyl, isopropyl, butyl, s-butyl, t-butyl, butyryl, cyclohexyl, and cyclohexenyl groups. The aryl substituent in an aryl group comprising a substituent desirably has 6 to 20, preferably 6 to 14, total carbon atoms. Examples include phenyl, naphthyl, anthracenyl, methoxyphenyl, and chlorophenyl groups. Such substituent aryl groups may have preferably 6 to 40, more preferably 6 to 25, total carbon atoms. Specific examples include ethylphenyl, biphenyl, nonylphenyl, octylphenyl, fluorophenyl iodophenyl, triiodophenyl, methoxyphenyl, cyanophenyl, ethylnaphthyl, and iodonaphthyl groups.

R⁴ preferably represents phenyl group, iodophenyl group, triiodophenyl group, butylphenyl group, hexylphenyl group, octylphenyl group, biphenyl group, naphthyl group, or iodonaphthyl group; and more preferably represents an iodophenyl group, triiodophenyl group, hexylphenyl group, octylphenyl group, or iodonaphthyl group.

An optimal example of the above polymer includes a copolymer of compound A below with an acrylic acid ester or methacrylic acid ester.

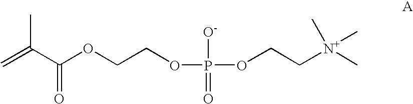

An example of a method of synthesizing the polymer is placing a monomer compound having the structural unit of the polymer in a reaction vessel along with a solvent, and suitably heating the mixture in the presence of an initiator under a nitrogen atmosphere. In the case of copolymerization, copolymerization components in the form of monomer compounds having the structural units of the polymer are placed together and a polymerization reaction is conducted in a similar manner to the above.

The solvent employed in polymerization need only be capable of dissolving the monomer compound employed. Examples of the solvent include water, methanol, ethanol, propanol, butanol, tetrahydrofuran, acetonitrile, acetone, benzene, toluene, dimethylformamide, and mixtures of any of these solvents.

The initiator employed in polymerization need only be a common radical initiator; examples include aliphatic azo compounds such as 2′-azobisisobutyronitrile and azobismalenonitrile; and organic peroxides such as benzoyl peroxide, lauroyl peroxide, ammonium persulfate, and potassium persulfate.

Examples of the paramagnetic metal compound include iron oxides and paramagnetic metal complex compounds.

An example of an iron oxide includes the ferrite represented by formula (X) below:

(MO)nFe₂O₃ (X)

(wherein M represents a divalent metal and n represents the integer 0 or 1). Examples of the divalent metal represented by M include magnesium, calcium, manganese, iron, nickel, cobalt, zinc, strontium, and barium. M preferably represents divalent iron. The molar ratio of M/Fe can be determined based on the stoichiometric composition of the ferrite selected. Salts of the above may also be employed; the type of salt is not specifically limited, but chloride salts, bromide salts, or sulfates are preferable. These salts may be employed in the form of powders, dispersions, or the like. The iron oxide employed in the present invention is preferably in the form of a magnetic iron oxide crystal microparticle, such as magnetite or maghemite.

Further, examples of the iron oxide include magnetic iron oxide, gamma-iron oxide, and particles coated with other iron/metal oxides of high magnetic susceptibility. An example is magnetite, which is a T2 intensifying imaging agent that shortens the transverse relaxation time (T2) of protons. Specific examples include superparamagnetic iron oxide microparticles (superparamagnetic iron oxide: SPIO) and ultrasmall superparamagnetic iron oxide microparticles (ultrasmall superparamagnetic iron oxide: USPIO).

The paramagnetic metal complex compound is a complex compound comprised of paramagnetic metal ions of a lanthanoid series element, or some other transition metal, chemically bonded to a chelating compound.

Various paramagnetic metals can be employed as the metal atoms of the paramagnetic metal complex compound. Preferable examples include the lanthanoid series elements of atomic numbers 57 to 70, particularly gadolinium (Gd), dysprosium (Dy), ytterbium (Yb), praseodymium (Pr), neodymium (Nd), samarium (Sm), terbium (Tb), holmium (Ho), and erbium (Er). Additional examples in the form of other metals include transition metals such as chromium (Cr), manganese (Mn), iron (Fe), cobalt (Co), nickel (Ni), and copper (Cu). Preferable examples include Gd³⁺, Dy³⁺, Mn²⁺, and Fe³⁺, with Gd³⁺ being optimal.

The chelating compound employed to prepare the paramagnetic metal complex compound is not specifically limited other than that it be suitably lipophilic to a degree permitting the formation of a complex with paramagnetic metal atoms and enclosure by the polymer. For example, any of the various useful chelating compounds proposed thus far as macrocyclic chelating agents (for example, WO9008134, the disclosure of which is expressly incorporated by reference herein in its entirety) may be employed.

A cyclic or chainlike polyaminopolycarboxylic acid having an active amino group as a crosslinking chain, containing a bifunctional structure having the ability to capture metal ions and form complexes, is preferable as the chelating compound. For example, diethylenetriaminepentaacetic acid (DTPA) derivatives and salts thereof come to mind.

Specific examples include monoalkylamide DTPA, dialkylamide DTPA, monoarylamide DTPA, diarylamide DTPA, monoalkylester DTPA, dialkylester DTPA, monoarylester DTPA, diarylester DTPA, and alkylated DTPA. In these compounds, examples of the alkyl include alkyl groups with 120 carbon atoms and examples of the aryl include phenyl and naphthyl. The aryl may be substituted with an alkyl, a halogen atom, or the like.

Additional examples of the chelating compound include triethylenetetraaminehexaneacetic acid (TTHA), ethylenediaminetetraacetic acid (EDTA), 1,4,7,10-tetraazacyclododecane-1,4,7-10-tetraacetic acid (DOTA), N,N-ethylenebis[2-(2-hydroxyphenyl)glycine] (EHPG), 1,4,8,11-tetraazacyclotetradecane (Cyclam), NTA, HEDTA, BOPTA, NOTA, DO3A, HPDO3A, EOB-DTPA, TETA, HAM, DPDP, porphyrins, and their derivatives. EOB stands for “ethoxybenzyl.”

The paramagnetic metal atom ions and the chelating agent are chelation bonded by the usual methods. Examples of the resulting paramagnetic metal complex compounds include Gd-DTPA, Gd-EOB-DTPA, Yb-EOB-DTPA, Dy-EOB-DTPA, Mn-DTPA, Gd-BOPTA, Gd-DOTA, and Gd-HPDO3A.

The paramagnetic metal complex compounds may be employed singly or in combinations of 2 or more. They are not limited to the compounds included in the examples of chelating compounds set forth above.

A paramagnetic metal, or compound thereof, that is suitable as the magnetic resonance imaging agent of the present invention desirably satisfies the following conditions. In addition to possessing the physical and chemical properties permitting use as an imaging agent, it is desirably a compound that can be formulated in the form of an aqueous solution in such a manner as to contain paramagnetic metal atoms in a quantity of 0.01 mg or greater based on weight per mL of imaging agent. Further, it is desirably highly hydrophilic, does not exhibit a high osmotic pressure even at high concentrations, and permits the preparation of a highly stable imaging agent.

The magnetic resonance imaging agent of the present invention further comprises a ligand molecule.

The term “ligand molecule” refers to a molecule that provides information (stimulus) to the cells of the body, either from the exterior or from within the body, by binding to or interacting with proteins present in cells, particularly moieties known as receptors. The actions of ligands on receptors cause cells to exhibit various responses. Examples include the activation of endocytosis dependent on specific ligands, and the incorporation of substances into cells. Examples of common ligand molecules include the phospholipid group, membrane proteins, hormones, and cytokines.

A particularly preferable example of a ligand molecule includes a compound represented by general formula (2).

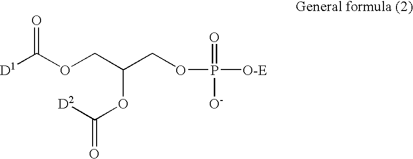

In formula (2), each of D¹ and D² is defined identically with R4 above. E is defined identically with A above.

Phosphatidic acid, phosphatidylethanolamine, phosphatidylserine, and phosphatidylinositol are particularly preferable as the ligand molecules. As has been reported in the J. Biol. Chem., 265, 5226 (1990), liposomes formed from phosphatidylcholine and phosphatidylserine are known to tend to cause the accumulation of macrophages via scavenger receptors.

The weight ratio of the ligand molecule to the above polymer (weight of ligand molecule: weight of polymer) is preferably from 10:90 to 50:50, more preferably from 15:80 to 40:60, and still more preferably, from 20:80 to 35:70.

The weight ratio of the polymer to the paramagnetic compound (weight of polymer: weight of paramagnetic metal compound) is preferably from 99.9:0.1 to 80:20, more preferably from 99.3:0.7 to 90:10, and still more preferably, from 99.6:0.4 to 90:10.

The above weight ratios are based on solid components, and do not include solvent.

The polymer preferably forms a particle with the paramagnetic metal compound. The particle preferably has the structure of a liposome, or is in the form of a macromolecular structure mimicking a liposome structure. Within the particle, the paramagnetic metal compound may be enclosed within the polymer, or the polymer may form a film with the paramagnetic metal compound on the particle. The diameter of the particle is preferably 4 to 400 nm, more preferably 4 to 200 nm.

The ligand molecule preferably forms the particle together with the polymer and the paramagnetic metal compound. Within the particle, the paramagnetic metal compound may be enclosed by both the ligand molecule and the polymer, or the ligand molecule may form a film with the polymer and the paramagnetic metal compound on the particle.

The magnetic resonance imaging agent of the present invention may be prepared by known methods.

Specifically, the polymer and solvent (concentration 10 to 30 weight percent) are charged to a reaction vessel and heated to 50 to 80° C. to form a solution. An aqueous solution of the paramagnetic metal compound is separately prepared (concentration 1 to 30 weight percent) and admixed with the polymer solution. The mixture is stirred for about 30 minutes, a ligand molecule aqueous solution is added to the mixed solution, and the mixture is stirred for another 10 minutes to obtain imaging agent particles.

Any solvent in which the polymer is soluble may be employed to dissolve the polymer. Examples include ethanol, propanol, butanol, tetrahydrofuran, acetonitrile, dimethylformamide, and mixed solvents thereof.

Although it is not intended to be bound by any specific theory, it is known that, in vascular diseases such as arteriosclerosis or restenosis after percutaneous transluminal coronary angioplasty (PTCA), vascular smooth muscle cells constituting tunica media of blood vessel abnormally proliferate and migrate into endosporium at the same time to narrow blood flow passages. Although triggers that initiate the abnormal proliferation of normal vascular smooth muscle cells have not yet been clearly elucidated, it is known that migration into endosporium and foaming of macrophages are important factors. It is reported that vascular smooth muscle cells then cause phenotype conversion (from constricted to composite type).

If the imaging agent of the present invention is used, the paramagnetic compound can be selectively taken up into the vascular smooth muscle cells abnormally proliferating under influences of foam macrophages. As a result, imaging becomes possible with high contrast between vascular smooth muscle cells of a lesion and a non-pathological site. Therefore, the imaging agent of the present invention can be suitably used particularly for MRI of vascular diseases. For example, imaging of arteriosclerotic lesion or restenosis after PTCA can be performed.

The imaging agent of the present invention can be stably formed with a particulate structure. Accordingly, the imaging agent of the present invention can be made to accumulate at tissue and disease sites of macrophage localization during use. Use of the imaging agent of the present invention permits the accumulation of more paramagnetic metal compound at macrophages than when employing a known technique such as a suspension or an oil emulsion.

Examples of tissues at which localization of macrophages is found that can be suitably imaged by the macromolecular structure of the present invention mimicking a liposome include blood vessels, the liver, lung cells, the lymph nodes, lymphoducts, and the renal epithelium. For some diseases, macrophages are known to assemble at disease sites. Examples of such diseases are tumors, arteriosclerosis, inflammation, and infection. Accordingly, the use of the imaging agent of the present invention permits specification of such disease sites. In particular, foamed macrophages that have absorbed large quantities of denatured LDL through scavenger receptors are known to accumulate in the initial stages of the formation of atherosclerotic lesions (Am. J. Pathol., 103, 181 (1981); and Annu. Rev. Biochem., 52, 223 (1983), the disclosures of which are expressly incorporated by reference herein in their entireties). Causing the imaging agent of the present invention to accumulate in such macrophages and conducting imaging by MRI permits the specification of the positions of initial arteriosclerotic lesions, which is difficult to achieve by other means.

The imaging method employing the imaging agent of the present invention is not specifically limited. For example, imaging can be conducted in the same manner as in imaging methods employing the usual MRI imaging agents by measuring changes in the T1/T2 relaxation times of water. The appropriate use of suitable metal ions permits use as a scintigraphy imaging agent, a X-ray imaging agent, a photoimaging agent, or a ultrasound contrast agent.

### EXAMPLES

The present invention is specifically described through embodiments below. However, the scope of the present invention is not limited to the embodiments presented below.

### Synthesis Example 1

To a reaction vessel were charged 6 weight parts of 2-(methacryloyloxy)ethylphosphorylcholine synthesized by consulting J. Chem. Soc., Perkin Trans. 1, 2000, 653-657, the disclosure of which is expressly incorporated by reference herein in its entirety; 14 weight parts stearyl methacrylate; and 90 weight parts of 1-propanol. To this was added 0.5 weight part of V-601 (made by Wako Pure Chemical Industries, Ltd.), and the mixture was reacted for 10 hours at 85° C. in a nitrogen atmosphere.

When the reaction had ended, the reaction solution was placed in acetone to reprecipitate the polymer, which was filtered and vacuum dried to obtain 15 weight parts of polymer A with a molecular weight of 68,000.

The molecular weight was measured by GPC. The measurement conditions were tetrahydrofuran/1-butanol=8/2, 5 mM LiCl, 0.1% (w/v) phosphoric acid, and a flow rate of 0.7 mL/min. Columns in the form of TSKgel-G2500H_{XL} (made by Toso) and TSKgel-GMH_{XL} (also made by Toso) were employed.

### Synthesis Example 2

To a reaction vessel were charged 8 weight parts of 2-(methacryloyloxy)ethylphosphorylcholine synthesized by consulting J. Chem. Soc., Perkin Trans. 1, 2000, 653-657; 12 weight parts stearyl methacrylate; and 90 weight parts of 1-propanol. To the mixture was added 0.5 weight part of V-601 (made by Wako Pure Chemical Industries, Ltd.), and the mixture was reacted for 10 hours at 85° C. in a nitrogen atmosphere.

When the reaction was completed, the reaction solution was placed in acetone to reprecipitate the polymer, which was filtered and vacuum dried to obtain 16 weight parts of polymer B with a molecular weight of 70,000.

The molecular weight was measured in the same manner as in Synthesis Example 1.

### Example 1

To a flask with a threaded neck were charged 0.15 weight part of polymer A and 0.8 weight part of n-propanol (made by Wako Pure Chemical Industries, Ltd.) and the mixture was heated to 60° C. and dissolved. A 0.02 weight part quantity of diethylenetriaminepentaacetic acid gadolinium (III) dihydrogen salt hydrate (made by Aldrich) was dissolved in 3.2 weight parts of pure water, the aqueous solution was added to the polymer solution, and the mixture was stirred for 30 minutes at 60° C.

Subsequently, an aqueous solution of 0.025 weight part of phosphatidic acid in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 1.

The solid component concentration of the particle dispersion was determined by weighing particle dispersion 1 in an aluminum dish, drying it on a hotplate (150° C., 120 minutes), and weighing the solid component. The quantity of gadolinium present in the particle dispersion was measured by ICP-MS (using an HP-4500 made by Agilent Technologies). The average diameter of the particles present in the particle dispersion was measured with a particle size measuring device (UPA-EX150 made by Nikkiso Co., Ltd.). The data are given in Table 1.

### Example 2

To a flask with a threaded neck were charged 0.15 weight part of polymer A and 0.8 weight part of n-propanol (made by Wako Pure Chemical Industries, Ltd.) and the mixture was heated to 60° C. and dissolved. A 0.02 weight part quantity of diethylenetriaminepentaacetic acid gadolinium (III) dihydrogen salt hydrate (made by Aldrich) was dissolved in 3.2 weight parts of pure water, the aqueous solution was added to the polymer solution, and the mixture was stirred for 30 minutes at 60° C.

Subsequently, an aqueous solution of 0.05 weight part of phosphatidic acid in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 2.

The solid component concentration, quantity of gadolinium, and average particle diameter of the particle dispersion were measured by the same methods as in Example 1. The data are given in Table 1.

### Example 3

To a flask with a threaded neck were charged 0.15 weight part of polymer A and 0.8 weight part of n-propanol (made by Wako Pure Chemical Industries, Ltd.) and the mixture was heated to 60° C. and dissolved. A 0.02 weight part quantity of diethylenetriaminepentaacetic acid gadolinium (III) dihydrogen salt hydrate (made by Aldrich) was dissolved in 3.2 weight parts of pure water, the aqueous solution was added to the polymer solution, and the mixture was stirred for 30 minutes at 60° C. Subsequently, an aqueous solution of 0.025 weight part of phosphatidylserine in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 3.

The solid component concentration, quantity of gadolinium, and average particle diameter of the particle dispersion were measured by the same methods as in Example 1. The data are given in Table 1.

### Example 4

To a flask with a threaded neck were charged 0.15 weight part of polymer A and 0.8 weight part of n-propanol (made by Wako Pure Chemical Industries, Ltd.) and the mixture was heated to 60° C. and dissolved. A 0.02 weight part quantity of diethylenetriaminepentaacetic acid gadolinium (III) dihydrogen salt hydrate (made by Aldrich) was dissolved in 3.2 weight parts of pure water, the aqueous solution was added to the polymer solution, and the mixture was stirred for 30 minutes at 60° C.

Subsequently, an aqueous solution of 0.05 weight part of phosphatidylserine in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 4.

The solid component concentration, quantity of gadolinium, and average particle diameter of the particle dispersion were measured by the same methods as in Example 1. The data are given in Table 1.

### Example 5

To a flask with a threaded neck were charged 0.15 weight part of polymer A and 0.8 weight part of n-propanol (made by Wako Pure Chemical Industries, Ltd.) and the mixture was heated to 60° C. and dissolved. A 0.02 weight part quantity of diethylenetriaminepentaacetic acid gadolinium (III) dihydrogen salt hydrate (made by Aldrich) was dissolved in 3.2 weight parts of pure water, the aqueous solution was added to the polymer solution, and the mixture was stirred for 30 minutes at 60° C.

Subsequently, an aqueous solution of 0.025 weight part of phosphatidylinositol in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 5.

The solid component concentration, quantity of gadolinium, and average particle diameter of the particle dispersion were measured by the same methods as in Example 1. The data are given in Table 1.

### Example 6

To a flask with a threaded neck were charged 0.15 weight part of polymer A and 0.8 weight part of n-propanol (made by Wako Pure Chemical Industries, Ltd.) and the mixture was heated to 60° C. and dissolved. Within a flask with a threaded neck were dissolved 0.15 weight part of polymer B and 0.02 weight part of diethylenetriaminepentaacetic acid gadolinium (III) dihydrogen salt hydrate (made by Aldrich) in 3.2 weight parts of pure water, the aqueous solution was added to the polymer solution, and the mixture was stirred for 30 minutes at 60° C.

Subsequently, an aqueous solution of 0.05 weight part of phosphatidylinositol in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 6.

The solid component concentration, quantity of gadolinium, and average particle diameter of the particle dispersion were measured by the same methods as in Example 1. The data are given in Table 1.

### Example 7

To a flask with a threaded neck were charged 0.15 weight part of polymer B, 0.02 weight part of diethylenetriaminepentaacetic acid gadolinium (III) dihydrogen salt hydrate (made by Aldrich), and 0.8 weight part of butanol (made by Wako Pure Chemical Industries, Ltd.) and the mixture was heated to 60° C. and dissolved. A 3.2 weight part quantity of pure water was added, and the mixture was stirred for 30 minutes at 60° C. Subsequently, an aqueous solution of 0.05 weight part of phosphatidylethanolamine in 10 weight parts of pure water was added, after which 5.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 7.

The solid component concentration, quantity of gadolinium, and average particle diameter of the particle dispersion were measured by the same methods as in Example 1. The data are given in Table 1.

### Reference Example 1

To a flask with a threaded neck were charged 0.15 weight part of polymer A, 0.02 weight part of diethylenetriaminepentaacetic acid gadolinium (III) dihydrogen salt hydrate (made by Aldrich), and 0.8 weight part of propanol (made by Wako Pure Chemical Industries, Ltd.) and the mixture was heated to 60° C. and dissolved. A 3.2 weight part quantity of pure water was added, and the mixture was stirred for 30 minutes at 60° C. Subsequently, 15.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 8.

The solid component concentration, quantity of gadolinium, and average particle diameter of the particle dispersion were measured by the same methods as in Example 1. The data are given in Table 1.

### Reference Example 2

To a flask with a threaded neck were charged 0.15 weight part of polymer B, 0.02 weight part of diethylenetriaminepentaacetic acid gadolinium (III) dihydrogen salt hydrate (made by Aldrich), and 0.8 weight part of propanol (made by Wako Pure Chemical Industries, Ltd.) and the mixture was heated to 60° C. and dissolved. A 3.2 weight part quantity of pure water was added, and the mixture was stirred for 30 minutes at 60° C. Subsequently, 15.8 weight parts of pure water were added, and the mixture was stirred for 10 minutes at 60° C. The aqueous solution was subjected to gel filtration (Sephodex G-25M: made by GE Healthcare) and centrifugally separated (9,000 rpm, 60 minutes), after which the supernatant was collected, yielding particle dispersion 9.

The solid component concentration, quantity of gadolinium, and average particle diameter of the particle dispersion were measured by the same methods as in Example 1. The data are given in Table 1.

TABLE 1

| | | Solid | |
|---|---|---|---|
| | | component | Quantity of |
| | Average | concentration | gadolinium ions |
| | particle | (weight | (μg Gd/mg |
| | diameter (nm) | mg/mL) | particles) |
| Example 1 | 68 | 3.8 | 4.3 |
| Example 2 | 157 | 4.7 | 3.6 |
| Example 3 | 166 | 5.5 | 2.2 |
| Example 4 | 130 | 4.8 | 5.3 |
| Example 5 | 75 | 4.2 | 7.3 |
| Example 6 | 210 | 3.4 | 6.6 |
| Example 7 | 195 | 4.1 | 5.1 |
| Reference | 65 | 4.5 | 4.8 |
| Example 1 | | | |
| Reference | 180 | 3.9 | 5.9 |
| Example 2 | | | |

### (Evaluation of Imaging Agent Particles)

### Preparation of Cells for Use in Evaluation

(THP-1) cells (prepared by DS Pharma Biomedical Co., Ltd.: human monocyte strain) were incubated using RPMI1640 medium containing 10 percent FBS at 37° C. with 5 percent CO₂ to induce macrophage-like cell differentiation. The culture plate employed had 6 wells with a capacity of 2.5 mL. A2.5 mL quantity of a dispersion of THP-1 cells (4.0×10⁵ cells/mL) in 10 ng/mL of PMA (phorbol ester) was added to each well. When 7 days had passed, the medium was replaced with 1 mL of RPMI1640 containing no FBS and the cells were incubated for 24 hours.

### Imaging Agent Particle (Example Sample) Uptake (Quantification) Test

From each of the wells in the plate was removed 250 microliter of medium. The removed medium was replaced with 250 microliter of sample of each of the various example, and incubation was conducted for 24 hours at 37° C. and 5 percent CO₂. The cells were washed three times with physiological saline, and then lysed with 0.1 percent SDS. The quantity of gadolinium present in the cell lysate solution was measured by ICP-MS (HP-4500 made by Agilent Technologies) and the uptake rate was calculated by the following equation. The results are given in Table 2.

Cell particle uptake rate=(quantity of gadolinium present in cell lysate solution)/(quantity of gadolinium added to cells)

TABLE 2

| | Quantity of | | |
|---|---|---|---|
| | Gd added to | Quantity of Gd | Uptake |
| | cells | detected after | rate |
| | (ppm) | 24 hours (ppb) | (%) |
| Example 1 | 1.63 | 3.5 | 0.21 |
| Example 2 | 1.69 | 7.6 | 0.45 |
| Example 3 | 1.21 | 5.2 | 0.43 |
| Example 4 | 2.54 | 21.8 | 0.86 |
| Example 5 | 3.07 | 42.5 | 1.38 |
| Example 6 | 2.24 | 16.3 | 0.73 |
| Example 7 | 2.09 | 21.5 | 1.03 |
| Reference | 2.16 | 2.8 | 0.13 |
| Example 1 | | | |
| Reference | 2.30 | 3.1 | 0.13 |
| Example 2 | | | |

From Table 2, it will be understood that the quantity of paramagnetic metal incorporated by macrophage-like cells was significantly higher when ligand molecule-modified particles were employed as the polymer particles. It was even higher when phosphatidylserine or phosphatidylinositol was employed as the ligand molecule.

In particular, the ratio was significantly higher when the molar ratio of the structural unit represented by general formula (1): the molar ratio of structural units represented by general formula (2) was from 20:80 to 30:70.

These results indicate that the imaging particle of the present invention is readily incorporated by macrophages. The imaging agent of the present invention is thought to be useful as an imaging agent for inflammatory diseases (including arteriosclerotic lesions) exhibiting surplus macrophages.

### INDUSTRIAL APPLICABILITY

The present invention provides a magnetic resonance imaging agent that is retained well in the blood and accumulates well in diseased areas.

What is claimed is:

1. A magnetic resonance imaging agent which comprises: a polymer comprising the structural unit represented by the following formula (I) and the structural unit represented by the following formula (II) in a molar ratio of 5 to 80:20 to 95; a paramagnetic metal compound; and a ligand molecule: 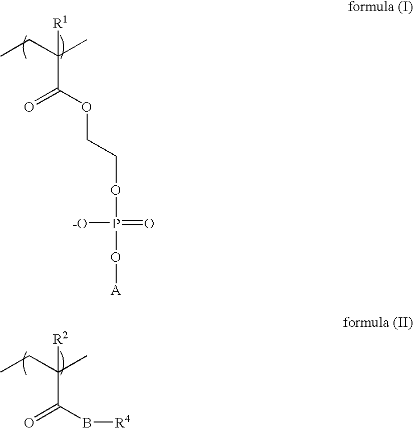
wherein R¹ represents hydrogen atom or methyl group; R² represents a hydrogen atom or a methyl group; A represents a group represented by one of the following formulas: 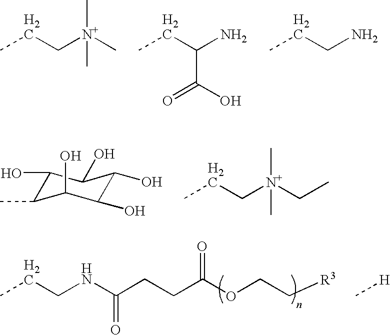
wherein the dotted line represents the O-A bond portion in formula (I); R³ represents hydroxyl group, methyloxy group, ethyloxy group, or phenyloxy group; and n represents an integer of 1 to 100; and B represents oxygen atom, sulfur atom, —CH₂—, or —NH—; and R⁴ represents hydrogen atom, an optionally substituted alkyl group, or an optionally substituted aryl group.

2. The magnetic resonance imaging agent according to claim 1, wherein the ligand molecule is the compound represented by general formula (2): 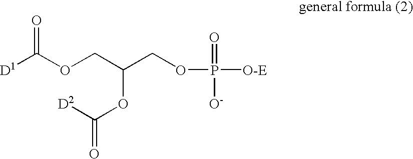
wherein each of D¹ and D² independently represents hydrogen atom, an optionally substituted alkyl group, or an optionally substituted aryl group; and E represents a group represented by one of the following formulas: 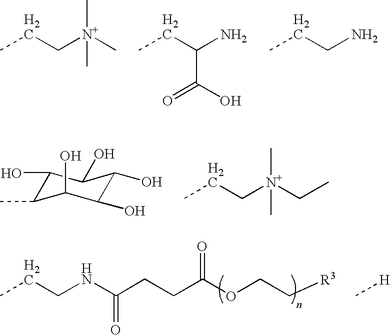
wherein the dotted line represents the O-A bond portion in formula (I); R³ represents hydroxyl group, methyloxy group, ethyloxy group, or phenyloxy group; and n represents an integer of 1 to 100.

3. The magnetic resonance imaging agent according to claim 1, wherein the ligand molecule is at least one member selected from the group consisting of phosphatidic acid, phosphatidylcholine, phosphatidylserine, phosphatidylethanolamine, and phosphatidylinositol.

4. The magnetic resonance imaging agent according to claim 1, wherein the weight ratio of the ligand molecule and the polymer is from 10:90 to 50:50.

5. The magnetic resonance imaging agent according to claim 1, comprising a particle having a diameter of 4 to 400 nm comprising the polymer and a paramagnetic metal compound.

6. The magnetic resonance imaging agent according to claim 1, wherein the polymer is a copolymer of compound A below and an acrylic acid ester or a methacrylic acid ester.

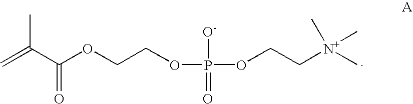

7. The magnetic resonance imaging agent according to claim 1 wherein the paramagnetic metal compound is iron oxide or a metal complex compound.

8. The magnetic resonance imaging agent according to claim 1, wherein the paramagnetic metal compound is a gadolinium metal complex compound.

9. The magnetic resonance imaging agent according to claim 1, which is used to image localized tissue or a diseased area in which the presence of macrophages or smooth muscle cells is pronounced.

10. The magnetic resonance imaging agent according to claim 9, wherein the localized tissue or diseased area in which the presence of macrophages or smooth muscle cells is pronounced is selected from the group consisting of a tumor, a site of inflammation, or a site of infection.

11. The magnetic resonance imaging agent according to claim 1, which is used to image vascular disease.

12. The magnetic resonance imaging agent according to claim 1, which is used to image an arteriosclerotic lesion.
